(19) United States
(12) Patent Application Publication
(10) Pub. No.: US 2014/0080907 A1
(43) Pub. Date: Mar. 20, 2014

(54) LIVER REGENERATION ACCELERATOR COMPRISING BETAINE

(71) Applicant: SNU R&DB FOUNDATION, Seoul (KR)

(72) Inventor: Young Chul Kim, Gyeonggi-do (KR)

(73) Assignee: SNU R&DB FOUNDATION, Seoul (KR)

(21) Appl. No.: 14/088,889

(22) Filed: Nov. 25, 2013

Related U.S. Application Data
(63) Continuation of application No. 13/392,971, filed on Feb. 28, 2012.

(30) Foreign Application Priority Data
Aug. 28, 2009 (KR) 10-2009-0080562

(51) Int. Cl. A61K 31/205

(52) U.S. Cl. CPC A61K 31/205
USPC 514/556; 562/575

(57) ABSTRACT

The present invention relates to betaine or a composition comprising the same for accelerating liver regeneration, more particularly, a therapeutic pharmaceutical composition comprising betaine as an active ingredient for promoting the recovery of injured or resected liver to the normal liver. The most effective treatment for chronic liver disease, particularly in the terminal stage, is hepatectomy and liver transplantation. In Living Donor Liver Transplantation (LDLT) that has been increasing since 1990, rapid regeneration of liver tissues is essential for both donor and recipient to inhibit complications and thus for the success of the treatment. Betaine accelerates the recovery and growth of injured liver to the normal liver, so that it can be effectively used for the successful LDLT or therapeutic hepatectomy.

### CROSS-REFERENCE TO RELATED APPLICATIONS

The present application is a continuation of U.S. application Ser. No. 13/392,971 filed on Feb. 28, 2012, which application is a national phase entry under 35 U.S.C. §371 of International Application No. PCT/KR2010/005637 filed Aug. 24, 2010, which claims priority from Korean Patent Application No. 10-2009-0080562 filed Aug. 28, 2009, all of which are hereby incorporated herein by reference.

### TECHNICAL FIELD

The present invention relates to betaine or a composition comprising the same that promotes regeneration of injured liver, particularly resected liver into normal liver in mammals including human.

### BACKGROUND ART

The liver is called ‘the silent organ’. It is because the liver can perform vital metabolic functions necessary for survival only with 15-20% of the liver remaining. Such a characteristic of the liver makes it difficult to diagnose the liver disease in early stages, and even after the diagnosis has been made, a liver patient frequently neglects it. According to the report issued by Statistics Korea, Republic of Korea, liver disease takes the 5th place in the list of leading causes of death in male (23.8 people per 100,000) and 10th place in female (7.8 people per 100,000) in 2007. Combining both male and female, liver disease takes the 8th place in the list of leading causes of death in Korean people. When hepatoma is included in the survey, overall liver disease is the number 3 cause of death only next to cerebrovascular disease and heart disease. In particular, among males at the age of 40s-50s which is the period for them to live a most active life, liver disease is the leading cause of death, suggesting that the management and treatment of liver disease is a very important and urgent matter along with the development of a novel therapeutic agent.

Liver injury can be induced by various insults such as ethanol, drugs, toxic chemicals, Hepatitis B and C virus, cholestasis, and autoimmunity. Liver disease generally progresses from fatty liver to hepatitis, hepatic fibrosis and hepatic cirrhosis. Fatty liver itself is not a disease and is a rather reversible symptom that can be cured spontaneously once the casual factor is eliminated. However, if the excessive accumulation of fat in liver tissues continues, steatohepatitis might be induced, which results in liver cell necrosis and regeneration, leading to hepatic fibrosis due to the increase of ECM (extracellular matrix) components. Once the liver is injured to a certain degree, destruction and regeneration of liver cells take place repeatedly regardless of the cause of liver injury and then regenerating nodules are formed, leading to the irreversible hepatic cirrhosis. Particularly, 20-30% of liver cirrhotic patients progress ultimately to hepatoma. There are two important factors involved in irreversible hepatic cirrhosis. One of them is the excessive deposition of ECM as consequence of chronic liver damage and the other is the loss of liver function due to the decrease of liver regeneration capacity.

The best treatment for a liver patient in the final stage is liver transplantation. However, the waiting recipients outnumber the donors greatly. To overcome this problem, a few dramatic methods have been proposed. One of them is the Living Donor Liver Transplantation (LDLT), which greatly reduces the gap between the number of waiting recipients and the number of organ donors. By this method, the death rate of pediatric patients in the waiting of liver transplantation reduced to almost “0” (de VIIIe et al., Chirurgie, 122; 83, 1997). The LDLT among adults has also been increased remarkably (Trotter et al., N. Engl. J. Med. 346; 1074, 2002). Currently 99% of liver transplantation performed in Japan is the LDLT (Nagai et al., Br. J. Surg. 96; 437, 2009).

Partial hepatectomy has also been accepted as the only fundamental standard treatment for hepatoma patients. However, excessive liver tissue resection may result in the loss of liver functions. In a patient with normal liver regeneration capacity, temporary hepatic failure can be quickly overcome. However, in a chronic liver patient with limited liver regeneration capacity, hepatic failure may persist, which might result in fatal complications. The death rate of hepatocellular carcinoma (HCC) patients resulting from the loss of liver functions after hepatectomy is still as high as 60-90% (Redaelli et al., World J Surg, 26; 1126, 2002).

Liver regeneration after partial hepatectomy is regulated by a series of complicate processes that convert the cell cycle from quiescent state to proliferative state. Various growth factors provide signals to stimulate or inhibit liver proliferation to control the processes (Michalopoulos, Science, 276; 60, 1997). Tissue necrosis factor α (TNF-α) and interleukin 6 are involved in the first stage of hepatocyte differentiation, and hepatocyte growth factor (HGF) and transforming growth factor α (TGF-α) promote the differentiation, and finally, TGF-β and activin inhibit the cell growth to terminate liver regeneration (Mariuchi et al., Biochem Biophys Res Commun 280; 368, 2001). However, in a patient with progressive fibrosis, the regeneration capacity is reduced significantly after hepatectomy (Kawasaki et al., Gastroenterology, 102; 1351, 1992; Andrian et al., J Surg Res, 89; 184, 2000), which reduces the success rate of treatment via liver transplantation. Recently, fatty liver has also been reported as an important factor that adversely affects the prognosis of a liver resected patient (Vetelainen et al., Ann Surg, 245; 20, 2007; McCormack et al., Ann Surg, 245; 923, 2007). It is suggested that the mechanism involved herein is the lipid peroxidation in fatty liver and Kupffer cell-mediated immune response that inhibit the growth of normal liver tissues (Vetelainen et al., Ann Surg, 245; 44, 2007).

Regeneration of resected liver to normal liver is a basic requirement for the successful transplantation to the donors as well. In the case of LDLT, it is important to leave enough volume of liver in the donor to prevent hepatic failure and at the same time, the recipient should receive a sufficient mass for successful treatment. Complications may occur for both liver tissue donor and recipient. In worst cases, even the donor may not survive (Surman, N Engl J Med, 346; 1038, 2002). It is not easy to calculate the risk for donor but it is presumed to be approximately 0.5% in Western European countries affiliated in European Liver Transplant Registry (ELTR) (Adams, ELTR, 2002). Compared with the risk rate of kidney donor (0.013%), this is a 40 times greater risk rate. In Asian Regional Center, 16% of 1508 donors experienced complications, 1% of them needed re-surgery, and at least one case of mortality was reported (Lo, Transplantation, 75(Suppl); S12, 2003). In USA, 14.5% of 449 donors experienced complications, 4.5% of them needed re-surgery, and one mortality case was reported (Brown et al., N Engl J Med, 348; 818, 2003). In conclusion, proper regeneration to normal liver may be the most important factor that determines the success of treatment for both simple partial hepatectomy patients (hepatoma patients and LDLT donors) and LDLT recipients.

Betaine represented by formula 1 is another name of trimethylglycine, which has a quaternary ammonium structure characterized by amphoterism in a neutral aqueous solution.

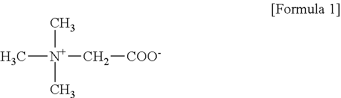

Betaine is widely distributed in plants and animals, and is synthesized by irreversible oxidation of choline in vivo.

Betaine is effective in treating homocysteinemia, the genetic disease characterized by the increase of homocysteine levels in urine and blood. Betaine has been approved by FDA, USA, to be used for treatment of homocysteinemia. It appears that betaine reduces blood homocysteine via enhancement of its methylation to methionine in liver, resulting in depletion of homocysteine both in liver and blood. WO98/19690 describes that betaine reduces the level of homocysteine that has been increased in blood of homocysteinemia patients. WO2000/51596 describes that glycine betaine, which is a pharmaceutical preparation containing betaine, is effective in the prevention and treatment of arterial/venous thromboembolic disease and coagulant disease. The U.S. Pat. No. 5,428,063 describes that when betaine is administered at a dose of at least 1,500 mg/kg/day, it inhibits fatty infiltration induced by ethanol or carbon tetrachloride, bringing the inhibitory effect on fatty liver generation. The present inventors also reported in Korean Patent Application No. 2008-0077400 that betaine may relieve the symptoms of hepatic fibrosis and cirrhosis. In addition, betaine can relieve dryness of mucous membrane (WO 1998/29090 and Korean Patent Laid-open No. 2000-0062425), and a pharmaceutical preparation comprising betaine and amine oxide can be used as an enveloped viral inhibitor (Korean Patent Laid-open No. 1992-0017649, Korean Patent No. 10-0221486). A substance containing pancreatine, betaine HCl, and dibasic calcium phosphate can be used as a feed additive for improving digestion efficiency (Korean Patent Laid-open No. 2002-0041162). However, there have been no reports made so far regarding the direct involvement of betaine in the progression of liver regeneration/liver growth or the usability of betaine in application of liver regeneration.

### DISCLOSURE

### Technical Problem

The present invention has been proposed to promote liver regeneration of injured liver. In another aspect, the present invention relates to provide a composition for the successful treatment of partial hepatectomy or Living Donor Liver Transplantation in chronic liver injury. In another aspect, the present invention relates to provide a composition for the improvement and treatment of complications after partial hepatectomy which comprises betaine, based on the confirmation that betaine can directly enhance the recovery of resected or injured liver to normal liver.

### Solution To Problem

The present invention provides a pharmaceutical composition for accelerating regeneration of injured liver, which comprises betaine represented by formula 1 as an active ingredient.

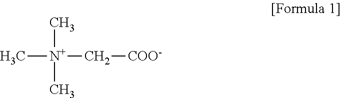

The said betaine herein includes betaine anhydrides, betaine hydrates, or hydrochlorides or any other pharmaceutically acceptable salts which can release betaine when they are dissolved in water. The betaine used in this invention can be extracted from natural sources by the method well-known to those in the art or synthesized, and the synthesis method or source is not limited.

The pharmaceutical composition of the present invention is preferably used for accelerating regeneration of partially resected liver.

The pharmaceutical composition of the present invention is preferably used for inhibiting complications of hepatectomy and for the recovery of injured liver to normal liver.

In a preferred embodiment of the present invention, ⅔ of the liver of a rat was excised and then betaine was supplied in drinking water during the recovery period. As a result, the growth of liver tissues was significantly enhanced and the hepatectomized liver reached the weight of the normal liver a week later in the above rat, unlike the rat supplied with filtered tap water as drinking water after partial hepatectomy. In the liver of the rat administered with betaine, cyclin D1 expressed in the restriction point of G1 phase of cell cycle and proliferating cell nuclear antigen (PCNA) expressed in S phase, which is DNA synthesis phase, are significantly increased. These results indicate that betaine has the effect of promoting recovery and regeneration of resected liver to normal liver. In conclusion, all the above results indicate that betaine is effective in regeneration of injured liver tissues, and thus the composition of the present invention comprising betaine can be used for the improvement or treatment of complications of hepatectomy by accelerating the recovery of partially resected liver or LDLT liver to normal liver.

The pharmaceutical composition of the present invention can be administered orally or parenterally and be used in general dosage forms of pharmaceutical formulation. That is, the composition for liver regeneration comprising betaine of the present invention can be prepared for oral or parenteral administration by mixing with generally used diluents or excipients such as fillers, extenders, binders, wetting agents, disintegrating agents and surfactants. Solid formulations for oral administration include tablets, pills, powders, granules and capsules. These solid formulations are prepared by mixing the said betaine with one or more suitable excipients such as starch, calcium carbonate, sucrose or lactose, gelatin, etc. In addition to the simple excipients, lubricants, for example magnesium stearate, talc, etc, can be used. Liquid formulations for oral administrations are suspensions, internal solutions, emulsions and syrups, and the liquid formulations can contain various excipients such as wetting agents, sweeteners, flavoring agent and preservatives in addition to generally used simple diluents such as water and liquid paraffin. Formulations for parenteral administration include injections, infusion solutions and suppositories, and sterilized aqueous solutions, nonaqueous excipients, suspension agents, emulsifying agents may be used to prepare the formulation. Nonaqueous excipients and suspension agents can include propylene glycol, polyethylene glycol, vegetable oil like olive oil, injectable ester like ethylolate, etc. Suppository bases can include Witepsol® (triglycerides of saturated vegetable fatty acid), macrogol, Tween® 61, cacao butter, laurin butter, glycerol, gelatin, etc.

The pharmaceutical composition of the present invention can additionally include other components necessary for regeneration of the liver. These components are, for example, amino acids composing proteins that form liver tissues, glucose, the energy source, etc. In particular, when a patient who may not eat a normal diet after the surgery, the pharmaceutical composition supplemented with the nutrients above can be added in infusion solutions, resulting in the increase of treatment effect. However, the pharmaceutical composition comprising betaine as an active ingredient and additional amino acids or glucose is only an example, and cannot limit the spirit and scope of the present invention in any way.

The effective dose of betaine, the active ingredient of the pharmaceutical composition of the present invention, is determined according to the administration method, age and weight of a patient, and severity of disease, etc. The dosage units can contain, for example, 1, 2, 3 or 4 individual doses or ½, ⅓ or ¼ of an individual dose. An individual dose contains the amount of active compound which is administered in one application and which usually corresponds to a whole, ½, ⅓ or ¼ of a daily dose.

The effective dosage of the pharmaceutical composition of the present invention can be determined according to absorption rate, inactivation rate and excretion rate of betaine, age, gender, health condition and severity of disease of a patient by those in the art. Preferably, the pharmaceutical composition can be administered at less than 2,000 mg/kg per day for an adult, and more preferably at 80-1,500 mg/kg per day.

Because of its endogenous nature, betaine is considered to be non-toxic. And the clinical use of betaine as a remedy for hyperhomocysteinemia has not shown any serious side effects.

The present invention also provides a health functional food comprising betaine as an active ingredient that can promote recovery and inhibit complications after the intensive post-care for hepatectomy (for example, for the patients after being discharged from hospital).

Betaine, the active ingredient of the present invention, can be added to a health functional food for the purpose of accelerating recovery and regeneration of injured liver regardless of the cause of liver disease. In the case of using betaine as a food additive, betaine can be added as it is or as mixed with other food or food ingredients according to the conventional method. The mixing ratio of active ingredients can be modified depending on the purpose of use (prevention, health enhancement or treatment). In general, to produce a health functional food or beverages, betaine, the active ingredient of the present invention is added preferably by up to 20 weight % and more preferably by up to 10 weight %. However, if long term administration is required for regulation of health conditions, the content can be lower than the above and higher content can be accepted as long as safety is guaranteed.

The food herein is not limited to a specific type of foodstuff. For example, betaine can be added to meats, sausages, breads, chocolates, candies, snacks, cookies, pizza, ramyuns, noodles, gums, dairy products including ice cream, soups, beverages, tea, drinks, alcohol drinks and vitamin complex, etc, and in a wide sense, almost every food applicable in the production of health functional food can be included.

The composition for health beverages of the present invention can additionally include various flavors or natural carbohydrates, etc, like other beverages. The natural carbohydrates can be one of monosaccharides such as glucose and fructose, disaccharides such as maltose and sucrose, polysaccharides such as dextrin and cyclodextrin, and glucose alcohols such as xylitol, sorbitol and erythritol. Besides, natural sweetening agents such as thaumatin and stevia extract, and synthetic sweetening agents such as saccharin and aspartame can be included as a sweetening agent.

In addition to the ingredients mentioned above, the health functional food of the present invention can include a variety of nutrients, vitamins, electrolytes, flavors, coloring agents, pectic acid and its salts, alginic acid and its salts, organic acid, protective colloidal viscosifiers, pH regulators, stabilizers, antiseptics, glycerin, alcohols, carbonating agents which used to be added to soda, etc. The health functional food of the present invention can also include natural fruit juices, fruit beverages and/or fruit flesh addable to vegetable beverages. All the mentioned ingredients can be used alone or in combination with other ingredients. The mixing ratio of those ingredients does not matter in fact, but in general, each can be added by 0.1-30 weight part per 100 weight part of betaine, the active ingredient of the present invention.

### Advantageous Effect

Betaine can be effectively used for both donor and recipient of Living Donor Liver Transplantation, and for partial hepatectomy to treat chronic liver injury because it can accelerate recovery and growth of injured liver to normal liver. The said effect of betaine is demonstrated when it is administered both before hepatectomy and also after hepatectomy.

Betaine also can be effectively used for promoting the recovery from liver disease due to its regeneration effect.

### DESCRIPTION OF DRAWINGS

The present invention is best understood with reference to the accompanying drawings, wherein:

FIG. 1a and FIG. 1b are diagrams illustrating the effect of betaine on the expression of PCNA (proliferating cell nuclear antigen) in the rat that had been partially hepatectomized.

FIG. 2 is a diagram illustrating the effect of betaine on the expression of cyclin D1 in the rat that had been partially hepatectomized.

### MODE FOR INVENTION

The present invention is illustrative as shown in the following Examples.

However, it will be appreciated that those skilled in the art, on consideration of this disclosure, may make modifications and improvements within the spirit and scope of the present invention.

Test Animal

Male Sprague Dawley rats were purchased from Central Lab. Animal Inc., Korea. Test animals were adapted to an animal facility where temperature was maintained at 22±2° C., humidity at 55±5%, and artificial illumination was provided for 12 hours a day, at least for a week before being used in the experiment. The rats weighed 230-260 g at the time when partial hepatectomy was performed. Filtered tap water and solid regular rat chow were provided freely. For the animal group that was supposed to be treated with betaine, betaine purchased from Sigma, USA, was dissolved in filtered tap water at the concentration of 1% and provided to the group as drinking water. Betaine was administered after partial hepatectomy was conducted. In some experiments, betaine containing drinking water was provided to the animal group from 2 weeks before partial hepatectomy to increase the effect of betaine.

Partial Hepatectomy

Partial hepatectomy was performed according to the method originally introduced by Higgins and Anderson (Arch Path, 12; 186, 1931). The rat was anesthetized lightly with ether, and then the middle line of abdomen was incised approximately 3 cm. First, the skin was cut and then abdominal muscle was incised carefully not to damage internal organs. The incised upper part was lifted up by using a pincette to cut the connective tissues connected with diaphragm supporting liver tissue using scissors. Light pressure was applied to the surrounding area of the incision line to expose median lobe and left lateral lobe taking about 70 volume % of the total hepatic lobes. Connective tissues of each hepatic lobe were carefully removed and tied with suture to close the blood vessel, followed by elimination of the exposed hepatic lobe. Muscles and skin of the incised area were stitched up with suture. The incised area was sterilized with 70% ethyl alcohol to prevent infection. For the control group, abdominal incision was simply performed and then stitched up (Sham).

Measurement of Hepatotoxic Parameters

Alanine aminotransferase (ALT) activity and aspartate aminotransferase (AST) activity were measured by the method of Reitman and Frankel (Amer J Clin Pathol 28; 56, 1957) using spectrophotometer. ALT, AST substrate solutions and an aliquot of serum were added to a test tube, followed by incubation at 37° C. Upon completion of the reaction, the color developing agent and 0.4 N NaOH were added thereto, followed by measurement of absorptivities at 520 nm. The activity of each enzyme was calculated based on the standard calibration curve generated by using pyruvate as a substrate.

Western Blotting Analysis

Cytosolic fraction was prepared from the excised liver using the standard method. The protein of cytosol was separated by SDS-PAGE, followed by electroblotting, and then transferred to nitrocellulose membrane. The membrane was treated with PBS-T buffer containing 5% nonfat milk at 4° C. for overnight. The nitrocellulose membrane was reacted with the primary antibody diluted with 5% bovine serum albumin. The primary antibodies used herein were mouse monoclonal anti-cyclin D1 serum (Santa Cruz Biotechnology, CA, U.S.A.) and rabbit polyclonal anti-human PCNA (Santa Cruz Biotechnology, CA, U.S.A.). After washing twice with PBS-T, the membrane was treated with the secondary antibody horseradish peroxidase-conjugated goat anti-rabbit IgG (Pierce Biotechnology, Rockford, Ill., U.S.A.). The result was obtained by scanning densitometry using microcomputer imaging device (Model M1, Imaging Research, St. Catharines, Canada).

The Effect of Betaine on Regeneration of Resected Liver

### Example 1

### The Effect of Betaine on Hepatotoxic Parameters and Liver Weight Increase in Resected Liver

Male rats were randomly divided into 4 groups of 4 rats each. Group 1, the control group, had laparotomy alone without damaging internal organs and then the opening was sealed (Sham group). Group 2 was treated with betaine after laparotomy (Sham+Betaine group). Group 3 had two-thirds (⅔) partial hepatectomy (PH group), and Group 4 was treated with betaine after ⅔ partial hepatectomy (PH+Betaine group). For the administration of betaine to the groups that had laparotomy only or partial hepatectomy, 1% betaine solution was provided as drinking water. For other groups, filtered tap water was provided as drinking water.

Changes of liver weight are summarized in Table 1.

TABLE 1
The effect of betaine on liver weight
increase in the rat after hepatectomy

| | Day | | | |
|---|---|---|---|---|
| Group | 0 | 1 | 2 | 7 |
| SH | 4.04 ± 0.08 | ND | ND | 3.37 ± 0.05a |
| | | | | (100%) |
| SH + | 4.04 ± 0.08 | ND | ND | 3.30 ± 0.10a |
| Betaine | | | | (98%) |
| PH | 4.04 ± 0.08 | 1.36 ± 0.03 | 1.79 ± 0.04 | 2.64 ± 0.01b |
| | | | | (78%) |
| PH + | 4.04 ± 0.08 | 1.47 ± 0.05 | 1.98 ± 0.05* | 3.17 ± 0.06*, a |
| Betaine | | | | (94%) |

In Table 1, the values on day 0 indicate the relative liver weight (liver/weight percentage) in the normal rats not having the surgery. Each group was composed of 4 rats except that 8 rats were used for the calculation for day 0 values. Value is mean±standard deviation. * Statistically significant difference between the group treated with betaine and the group without betaine treatment (Student's t-test, P<0.05). In the values of day 7, the group represented by a different alphabet showed a statistically significant difference (Oneway ANOVA followed by Neuman Keul Multiple range test, P<0.05). SH: Sham, PH: Partial hepatectomy. The number in parenthesis indicates % to the SH group.

As shown in Table 1, the relative liver weight (%) of the rat which was at the same age with the test animal without any surgery was 4.04±0.08. The relative liver weight of Sham group which had laparotomy alone was 3.37±0.05, one week after the surgery, suggesting that the body weight was increased rapidly during this period. The administration of betaine did not affect the relative liver weight. The liver of animals that had ⅔ partial hepatectomy was reduced to ⅓ in size, 24 hours after the surgery, but from then on the liver weight was gradually increased and recovered to 78% of the normal liver, 7 days after the hepatectomy. In the animal group that had been supplied with betaine containing drinking water after hepatectomy, the liver weight was increased rapidly from day 1, compared with the animals not treated with betaine, and the difference was statistically significant from day 2. On day 7, the liver weight was recovered to 94% of the normal liver, which was not statistically different from that of the Sham group.

At the end of the experiment, hepatotoxic parameters in serum were measured. The results are shown in Table 2. Hepatectomy itself or betaine administration did not affect serum AST and ALT activities. This result suggests that betaine dose not induce toxicity in the liver under the said conditions.

TABLE 2
Changes in serum hepatotoxic parameters in rats after hepatectomy

| Group | AST (units/ml) | ALT (units/ml) |
|---|---|---|
| SH | 79 ± 2 | 24 ± 2 |
| SH + Betaine | 83 ± 3 | 22 ± 2 |
| PH | 83 ± 2 | 24 ± 1 |
| PH + Betaine | 78 ± 11 | 27 ± 2 |

In Table 2, each group was composed of 4 rats. Value is mean±standard deviation. Statistically significant difference was not shown between different groups (Oneway ANOVA followed by Neuman Keul Multiple range test, P>0.05).

### Example 2

### The Effect of Betaine on Parameters for Liver Regeneration after Hepatectomy

During the process of liver regeneration, liver cells are converted from quiescent state to proliferative state. Cyclin D1 is expressed in the restriction point of G1 phase of cell cycle and PCNA is expressed in S phase, the DNA synthesis stage. Therefore, these two are accepted as important parameters for the progression of liver regeneration.

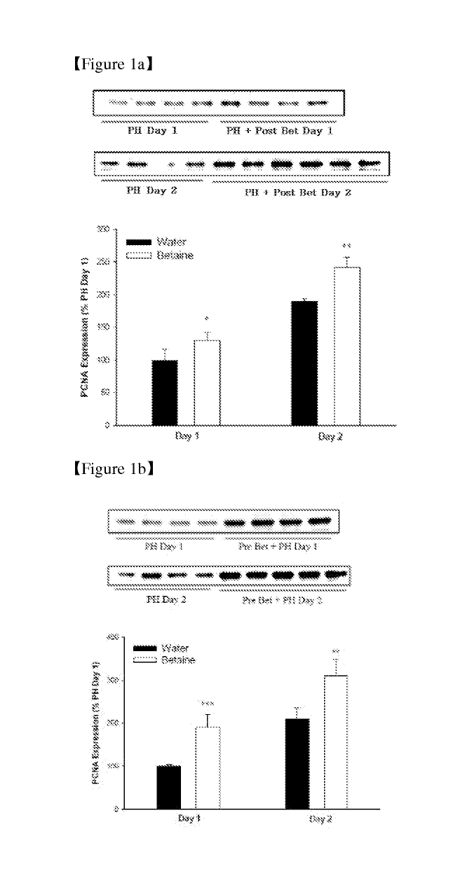

PCNA expression after hepatectomy is shown in FIG. 1a and FIG. 1b. FIG. 1a shows the result from the group provided with 1% betaine solution as drinking water after hepatectomy. Compared to the PCNA expression 24 hours after hepatectomy in rats supplied with filtered tap water as drinking water, PCNA expression in the above group was almost doubled in 48 hours after hepatectomy. In the group supplied with 1% betaine solution as drinking water after hepatectomy, PCNA expression was significantly increased both in 24 hours and in 48 hours as well, compared with the group treated with tap water as drinking water.

Usually, animals do not consume any feeds or drinking water right after hepatectomy. In order to demonstrate the efficacy of betaine more accurately, 1% betaine solution was provided from 2 weeks before hepatectomy and then PCNA expression was determined after the surgery. The result is shown in FIG. 1b. In the group which had been treated with betaine for 2 weeks before the surgery, PCNA expression was increased dramatically, compared with the group provided with tap water alone, which was 190% increase in 24 hours and 150% increase in 48 hours after hepatectomy.

In FIG. 1, each group was composed of 4 rats. Value is mean±standard deviation. In FIG. 1a and FIG. 1b, *, **, and *** indicate that there is a statistically significant difference between the rats treated with betaine and the rats without betaine treatment (Student's t-test, P<0.05, 0.01 and 0.001, respectively).

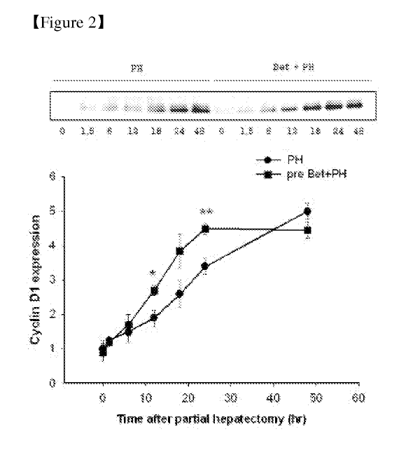

FIG. 2 illustrates the cyclin D1 expression in the recovery period after hepatectomy. In this experiment, betaine was administered to the rats in drinking water from 2 weeks before hepatectomy. In FIG. 2, the value at “0” hour represents the expression of cyclin D1 in the normal rats without hepatectomy but supplied with the betaine solution as drinking water. There was no significant difference in cyclin D1 expression between the normal rats supplied with filtered tap water as drinking water and the group treated with betaine. This result indicates that the administration of betaine itself does not affect the expression of cyclin D1 in normal liver that is not injured. After hepatectomy, the expression of cycline D1 was rapidly increased in the group provided with tap water, which was almost doubled at the 12th hour, 3.5 times higher at the 24th hour, and 5 times higher at the 48th hour. In the group treated with betaine, the expression of cyclin D1 was increased more rapidly after hepatectomy, which was 50% higher at the 12th hour than the group provided with tap water, which showed statistically significant differences. Such high cyclin D1 expression remained elevated for 24 hours after hepatectomy in the group treated with betaine. The difference between the two groups disappeared in 48 hours. This result indicates that the expression of cyclin D1 is increased more rapidly in the animals treated with betaine, compared with the group treated with tap water alone, in the early phase when the regeneration of injured liver is active after hepatectomy.

In FIG. 2, each group was composed of 4 rats. Value is mean±standard deviation. * and ** indicate that there is a statistically significant difference between the rats treated with betaine and the rats without betaine treatment (Student's t-test, P<0.05 and 0.01, respectively).

### Preparation Example 1

### Preparation of Pharmaceutical Formulations Containing Betaine

<1-1> Preparation of Syrups

Syrups containing betaine by 20% (weight/volume) as an active ingredient were prepared as follows. Betaine, saccharin and glucose were dissolved in 80 g of warm water. The mixture was cooled down, to which a mixture of glycerin, saccharin, flavoring, ethanol, sorbic acid and distilled water was added. Water was added to the mixture, making a total volume of 100 ml.

The constituents of the syrup are as follows.

| | | |
|---|---|---|
| Betaine | 20 | g |
| Saccharin | 0.8 | g |
| Glucose | 25.4 | g |
| Glycerin | 8.0 | g |
| Flavoring | 0.04 | g |
| Ethanol | 4.0 | g |
| Sorbic acid | 0.4 | g |

| | |
|---|---|
| Distilled water | proper amount |

<1-2> Preparation of Tablets

Betain (250 g) was mixed with 175.9 g of lactose, 180 g of potato-starch and 32 g of colloidal silicic acid. Gelatin solution (10%) was added to the mixture, which was then pulverized and filtered with 14-mesh sieve. The pulverized mixture was dried, to which 160 g of potato starch, 50 g of talc and 5 g of magnesium stearate were added to prepare tablets.

The constituents of the tablet are as follows.

| | | |
|---|---|---|
| Betaine | 250 | g |
| Lactose | 175.9 | g |
| Potato Starch | 180 | g |
| Colloidal silicic acid | 32 | g |
| 10% gelatin solution | | |
| Potato starch | 160 | g |
| Talc | 50 | g |
| Magnesium stearate | 5 | g |

<1-3> Preparation of Injectable Solutions

Betain (10 g), sodium chloride (0.6 g) and ascorbic acid (0.1 g) were dissolved in distilled water to make 100 ml of solution. The solution was put in a bottle and heated at 20° C. for 30 minutes for sterilization.

The constituents of the injectable solution are as follows.

| | |
|---|---|
| Betaine | 10 g |
| Sodium chloride | 0.6 g |
| Ascorbic acid | 0.1 g |
| Distilled water | proper amount |

### Preparation Example 2: Preparation of Health Functional Foods Containing Betaine

<2-1> Preparation of Foods

Foods containing betaine were prepared as follows.

1. Preparation of Spices for Cooking

Health enhancing spices for cooking were prepared with 20˜95 weight % of betaine according to the conventional method.

2. Preparation of Tomato Ketchup and Sauce

Health enhancing tomato ketchup or sauce was prepared by mixing 0.2˜1.0 weight % of betaine with tomato ketchup or sauce according to the conventional method.

3. Preparation of Flour Food

Betaine was added to flour at 0.5˜5.0 weight %. Health enhancing foods such as bread, cake, cookies, crackers and noodles were prepared with the flour mixture according to the conventional method.

4. Preparation of Soups and Gravies

Betaine was added to soups and gravies at 0.1˜5.0 weight %. Health enhancing meat products, soups and gravies were prepared with this mixture by the conventional method.

5. Preparation of Ground Beef

Health enhancing ground beef was prepared by adding betaine to ground beef at 1-10 weight % according to the conventional method.

6. Preparation of Dairy Products

Betaine was added to milk at 1˜5 weight %. Health enhancing dairy products such as butter and ice cream were prepared with the milk mixture according to the conventional method.

7. Preparation of Sun-Sik (Dried Ground Grain)

Brown rice, barley, glutinous rice and Yulmu (Job's tears) were gelatinized according to the conventional method, dried and pulverized to obtain 60-mesh powders. Black soybean, black sesame and wild sesame were steamed and dried according to the conventional method and pulverized to obtain 60-mesh powders. Sun-Sik was prepared by mixing the dry powders of the grains, seeds and betaine according to the below ratio.
Grains (brown rice: 30 weight %, Yulmu: 15 weight %, barley: 20 weight %),
Seeds (wild sesame: 7 weight %, black soybean: 8 weight %, black sesame: 7 weight %),
Betaine (3 weight %),
Ganoderma lucidum (0.5 weight %),
Rehmannia glutinosa (0.5 weight %)

<2-2> Preparation of Beverages

1. Preparation of Carbonated Beverages

Syrup was prepared by mixing betaine with sugar (5-10%), citric acid (0.05-0.3%), caramel (0.005-0.02%), vitamin C (0.1-1%) and purified water (79-94%). The syrup was sterilized at 85-98° C. for 20-180 seconds, and then mixed with cooling water at the ratio of 1:4. Carbon dioxide was injected thereto by 0.5-0.82% to prepare carbonated beverages containing betaine of the present invention.

2. Preparation of Health Beverages

Betaine was mixed with liquid fructose (0.5 weight %), oligosaccharide (2 weight %), sugar (2 weight %), salt (0.5 weight %), and water (75 weight %). After mixing completely, the mixture was sterilized instantly and filled in small containers such as glass bottles, PET bottles, etc, to prepare health beverages.

3. Preparation of Vegetable Juice

Health enhancing vegetable juice was prepared by adding 1-10 g of betaine to 1,000 ml of tomato or carrot juice according to the conventional method.

4. Preparation of Fruit Juice

Health enhancing fruit juice was prepared by adding 1-10 g of betaine to 1,000 ml of apple, grape or other fruit juice according to the conventional method.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for promoting regeneration of the partially resected liver using a composition comprising betaine represented by Formula 1 as an active ingredient:

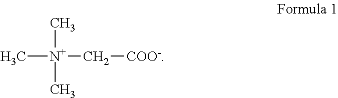

2. The method according to claim 1, wherein the betaine is in the form selected from the group consisting of betaine anhydrides, betaine hydrates, and pharmaceutically acceptable betaine salts.

3. The method according to claim 1, wherein the betaine is administered in the amount of from 80 to 1,500 mg/kg per day.

4. The method according to claim 1, wherein the composition further comprises one or more compounds selected from the group consisting of amino acids and glucose.
